(19) United States
(12) Patent Application Publication
(10) Pub. No.: US 2026/0315137 A1
(43) Pub. Date: Oct. 15, 2026

(54) Laparoscopic Applicator, and System for Laparoscopic Ablation

(71) Applicant: Stockert GmbH, Freiburg im Breisgau (DE)

(72) Inventors: Colin Corcoran, Schallstadt (DE); Fritz Koch, Freiburg (DE); Jonas Butz, Vogtsburg (DE); Patty Ky Ho, Merzhausen (DE)

(21) Appl. No.: 19/719,852

(22) Filed: Jun. 25, 2026

Related U.S. Application Data
(63) Continuation-in-part of application No. 18/944,629, filed on Nov. 12, 2024.

(30) Foreign Application Priority Data
Sep. 11, 2024 (DE) 10 2024 126 105.6

(51) Int. Cl. A61B 18/14; A61B 18/00; A61N 1/32

(52) U.S. Cl. CPC A61B 18/1445; A61B 18/1482; A61B 2018/00345; A61B 2018/00434; A61B 2018/00577; A61B 2018/00613; A61B 2018/00791; A61B 2018/00875; A61N 1/327

(57) ABSTRACT

The present disclosure describes a device for use in laparoscopic surgery, and a system for laparoscopic ablation. A method for extravascular, perivascular and/or perineural neuromodulation is also disclosed.

### CROSS-REFERENCE TO RELATED APPLICATIONS

The present application is a continuation-in-part application of, and claims priority to, U.S. Patent Application No. 18/944,629, filed on Nov. 12, 2024, which claims priority to German application no. 10 2024 126 105.6 filed on Sep. 11, 2024, the disclosures of which are incorporated by reference herein in their entireties.

### BACKGROUND AND SUMMARY

The present disclosure is classified in the field of minimally-invasive medical procedures, with special focus on the technique of ablation. It concentrates in particular on the irreversible electroporation (IRE) of tissue, an advanced method which is used in interventional medicine for the targeted treatment of tissue. This technique represents a significant advancement in medical therapy and is already being used in a targeted manner for the ablation of heart tissue, with particular consideration of tissue selectivity, reduced treatment times and the minimisation of risks of conventional treatment methods.

In recent years, the treatment of tissue by means of pulsed electrical fields has become established as an increasingly relevant clinical technique. The use of short high-voltage electrical pulses and the high electrical field strengths associated therewith, which act on the tissue, have, however, already been the subject of intensive research for more than four decades. This application method is categorised as a non-thermal procedure, since it is based on the delivery of short pulses with a high voltage amplitude, which generate between active electrode pairs a locally strong electrical field in the region of up to several thousand volts per centimetre. This field strength leads to the formation for a short time of pores in the cell membranes. If the electrical field exceeds a specific threshold value, which is required for the formation of pores in the lipid bilayers of the cell membranes, and if the tissue is exposed to that field beyond a critical period of time, the cells die as a result of apoptosis.

Pulsed field ablation has already been found to be a highly promising treatment method for the treatment of cardiac arrhythmias, such as, for example, atrial fibrillation. The neuromodulation of nerves by means of pulsed high-voltage electrical signals, on the other hand, is still the subject of numerous research projects. A considerable field of research covers renal denervation. Renal denervation is a method for suppressing increased sympathetic activity in order to treat high blood pressure or other cardiovascular diseases. The current therapeutic approach is a minimally-invasive intervention in which nerve conduction around the renal arteries is prevented with the aid of a special catheter. The catheter is inserted into the renal artery, and the nerve fibres running outside the artery are cauterised through the vessel wall by means of thermal or chemical ablation.

Although the minimally-invasive endovascular catheter-based approach for neuromodulation described above is regarded as a highly promising treatment method, some of the studies which have been carried out give rise to doubts about its effectiveness. Because, for example, the renal sympathetic nerve fibres are located outside the artery and thus cannot be reached directly with a catheter, a laparoscopy-based intervention for perivascular neuromodulation may represent a highly promising alternative procedure. In this manner, the nerve fibres can be brought directly into contact with a laparoscopic ablation instrument and thus the likelihood of successful treatment can be increased.

Irreversible electroporation (IRE) is essentially a non-thermal procedure which uses only a small amount of electrical energy and thus effects an increase in the tissue temperature by only a few °C. This distinguishes it significantly from conventional RF ablation (RF: radiofrequency), in which the tissue temperature increases by 20 to 70°C and cells are destroyed by heat. In IRE, biphasic or bipolar pulses are generally used, that is to say a combination of positive and negative electrical pulses, in order largely to avoid muscle contractions, which usually occur when DC voltage is used. These pulses can be applied between two bipolar electrodes of an applicator or between an applicator electrode and a body surface electrode, which is usually attached to the patient's back.

In order that the IRE pulses generate pores in the tissue, the electrical field strength E, defined by the pulses, at the tissue between a pair of at least two electrodes must exceed a tissue-dependent threshold value Eth. For example, the threshold value for cardiac cells is approximately 500 V/cm, while for bones it is 3000 V/cm. These differences in the threshold values allow the selective application of IRE in different tissues. In order to reach the required field strength, the voltage to applied to an electrode pair depends both on the target tissue and also on the distance between the electrodes and on the size of the electrodes themselves. These parameters likewise influence the thermal energy input during the ablation and thus the temperature peaks which can occur at the tissue to be treated. The applied voltages can reach several kilovolts and are thus significantly above the voltages of 10-200 V that are typical for thermal RF ablation.

Systems for the neuromodulation of nerves and especially for renal denervation that have become established on the market use a transvascular catheter-based approach, either by means of RF energy or ultrasound. However, a general disadvantage of transvascular neuromodulation is that, for cauterisation of the outer nerve fibres, ablation must be carried out through the vessel wall. In order to counteract this, an alternative treatment procedure provides for the use of laparoscopic applicators. These are inserted into the patient's torso and in this manner can address the nerves around the artery in a perivascular manner from outside. The required energy input into the tissue can thus be reduced, as a result of which on the one hand there is the promise of an increased likelihood of successful neuromodulation and at the same time the risk of damage to the vessel can be minimised.

Laparoscopic systems for such perivascular neuromodulation, for example for renal denervation, are known from EP 4335397 A1. That document describes an ablation device including an electrode unit, which is able to enclose a tube-shaped piece of tissue in order to denervate nerves via a thermal ablation procedure (for example radiofrequency ablation).

Animal studies demonstrated that there is a high variation regarding the anatomical configurations. The renal artery and the renal vein can run parallel or in close spatial proximity, and can be connected within the same perivascular connective tissue or fatty tissue in the region between the origin of the renal artery from the abdominal aorta and the entry of the renal vein, up to the renal hilum. However, because of the anatomical variation, the renal anatomy can also be presented with similar parallel or adjacent vessel conformations. In such conditions, this disclosure is relevant.

In case of connected vein and artery, a surgical separation of the vessels must be performed in order to expose only the renal artery and to achieve an effective lesion using specific devices such as HyperQure^{TM}.

In addition to that, surgical separation is required as veins are thin walled and more sensitive to adverse events when, specifically thermal energy is applied (e.g risk of stenosis) compared to artery walls.

However, due to the laparoscopic procedure, the field of view is limited and separating the vessels from each other is difficult. Further, even in open surgical procedures, exposing the renal artery is complex and time-consuming and requires specific training of the operating physicians as well as a high level of experience. Still further, the separation of the vessels is associated with a high risk of vascular injury or damage to surrounding tissue, and consequently with severe, potentially life-threatening bleeding that may affect the procedure.

There is a need for improved ablative approaches.

The present disclosure relates to laparoscopic ablation, in particular laparoscopy-based neuromodulation.

There are proposed for this purpose a device according to claim 1, a system according to claim 15 and a method according to claim 21.

According to a first aspect, a device for use in laparoscopic surgery is proposed. The device has a shaft. The shaft has a distal end and a proximal end. The device has a holding unit, which is arranged at the distal end of the shaft. The holding unit is adapted to hold and/or to release a tissue. The device has at least two electrodes integrated in/on/with the holding unit. The electrodes can be connected directly and/or permanently to the holding unit. The device has a force transmission unit which is arranged on/in the shaft and is connected, for example indirectly, to the holding unit and which is adapted to be moved relative to the shaft. The device has an electric cable, which is arranged in the shaft and is connected, in particular electrically conductively, to the at least two electrodes and is adapted to transmit an electrical signal that is to be received via the proximal end of the shaft to the at least two electrodes, in particular in order to carry out irreversible electroporation on/in/at a tissue that is being held.

The device can also be referred to as a laparoscopic applicator. The device can be used with a trocar, which is adapted to insert the shaft through/into a patient's torso.

The device can have an articulated arrangement. The articulated arrangement can be arranged between the holding unit and the distal end of the shaft. The articulated arrangement can be arranged at the distal end of the shaft and/or can be connected, in particular directly, thereto. The articulated arrangement can be adapted to connect the distal end of the shaft, in particular movably, to the holding unit. A movement of the force transmission unit can lead to a movement of the holding unit and/or to the tissue being held and/or released by means of the holding unit. A movement of the force transmission unit can lead to a movement of the articulated arrangement, which in turn leads/can lead to a movement of the holding unit and/or to the tissue being held and/or released.

The holding unit can be configured as a finger gripper, in particular as a two- or three-fingered gripper. The force transmission unit can be configured as a pull- and push-rod. A movement of the pull- and push-rod, in particular relative to the shaft, can lead to the opening or closing of the finger gripper in order to grip/clamp or release the tissue.

The device can have a longitudinal, transverse and vertical axis. The shaft can extend along the longitudinal axis. The pull- and push-rod can be adapted to move along the longitudinal axis. A movement of the pull- and push-rod along the/a, in particular longitudinal, axis leads to an opening movement of the finger gripper along a vertical and/or transverse axis, in particular in the vertical and longitudinal axial plane.

The fingers of the finger gripper can each have an inner face which, in particular when the finger gripper is in a closed state, can be oriented towards the tissue and/or can be in contact with a tissue that is being held. In each case one of the at least two, in particular ablation, electrodes, in particular one of a plurality of electrodes, can be integrated in/on at least one of at least two of the inner faces. At least two electrodes of the plurality of electrodes can be configured as ablation electrodes. At least three of the plurality of electrodes can be configured as measuring electrodes.

The holding unit can be configured as a suction unit. A movement of the force transmission unit can lead to an angling, for example the adjustment of any desired angle, in particular between 0° and 90° inclusive, in particular in a vertical and longitudinal axial plane, of the suction unit relative to the shaft. The suction unit can be adapted to suction a tissue and/or to fix itself to the tissue or to detach itself from the tissue.

The suction unit can have a suction cup. The suction unit can have at least one suction hole, which is arranged in the suction cup. The suction unit can have a suction channel, which is connected to the at least one hole. The suction unit can have a suction connection provided at one end of the suction channel. An external vacuum pump can be connected via the suction connection in order to generate a negative pressure in the suction cup and suction the tissue against/into the suction cup or, by switching off the external vacuum pump or reducing the negative pressure in the suction cup, to detach itself from the tissue.

The suction cup can be configured to be oval, in particular stadium-shaped or circular, along a vertical axis. The at least two, in particular ablation, electrodes can be arranged in the suction cup along the vertical axis and/or transverse to the vertical axis.

The suction cup can have a cup width and a cup length. The suction cup can have a flexible material. For example, the width of the suction cup can thus adapt itself (slightly) as soon as tissue is suctioned. The suction cup can thus adapt itself to a structure and/or shape of the tissue to be suctioned.

The suction cup can be configured to be oval, in particular stadium-shaped or circular, along a vertical axis. At least three, in particular measuring, electrodes can be arranged, for example in addition to the at least two electrodes, in the suction cup along the vertical axis and/or transverse to the vertical axis, in particular spaced apart from the at least two electrodes by a first electrode distance. The at least two/three electrodes can be arranged in the suction cup in an upper/lower region thereof along the vertical axis, while the at least three/two electrodes can be arranged in the suction cup in a lower/upper region thereof transverse to the vertical axis. Formulated in greater detail, the at least two electrodes (e.g., ablation electrodes) can be arranged in the suction cup in an upper region thereof along the vertical axis, while the at least three electrodes (e.g., measuring electrodes) can be arranged in the suction cup in a lower region thereof transverse to the vertical axis. Alternatively, the at least three electrodes (e.g., measuring electrodes) can be arranged in the suction cup in a lower region thereof along the vertical axis, while the at least two electrodes (e.g., ablation electrodes) can be arranged in the suction cup in an upper region thereof transverse to the vertical axis.

The holding unit can be configured as a clamshell gripper, in particular a double half-clamshell gripper. A movement of the force transmission unit can lead to an angling, in particular the adjustment of any desired angle, in particular between 0° and 90° inclusive, in particular in a transverse and longitudinal axial plane, of the clamshell gripper relative to the shaft, and/or to the opening and/or to the closing of the clamshell gripper.

The clamshell gripper can extend and/or have a maximum extent along a transverse axis. The clamshell gripper can be adapted to open or to close along a vertical axis. (The) at least (one of the) two electrodes can be integrated in an inside, in particular at least or solely, of a, for example each, clamshell, in particular can be oriented/extend along and/or transverse to a longitudinal axis. At least two, in particular ablation, electrodes can be integrated in an inside of each clamshell, in particular can be oriented/extend along and/or transverse to a longitudinal axis.

The clamshell gripper can extend and/or have a maximum extent along a transverse axis. The clamshell gripper can be adapted to open or to close along a vertical axis. At least (one of the) three, in particular measuring, electrodes can be integrated, for example in addition to the at least two, in particular ablation, electrodes, in an inside, in particular at least or solely, of one, for example each, clamshell, in particular can be oriented/extend along and/or transverse to a longitudinal axis and/or can be spaced apart from the at least two electrodes by a second electrode distance. At least three, in particular measuring, electrodes can be integrated in an inside of each clamshell, in particular can be oriented/extend along and/or transverse to a longitudinal axis.

The at least two/three electrodes can be arranged in the inside of the clamshell in an upper/lower region along the longitudinal axis, while the at least two/three electrodes can be arranged in a lower/upper region transverse to the longitudinal axis. Or, formulated in greater detail, the at least two electrodes (e.g., ablation electrodes) can be arranged in the inside of the clamshell in an upper region along the longitudinal axis, while the at least three electrodes (e.g., measuring electrodes) can be arranged in a lower region transverse to the longitudinal axis. Alternatively, the at least three electrodes (e.g., measuring electrodes) can be arranged in the inside of the clamshell in a lower region along the longitudinal axis, while the at least two electrodes (e.g., ablation electrodes) can be arranged in an upper region transverse to the longitudinal axis.

The force transmission unit can be configured as a first pull- and push-rod, which is arranged in the shaft and is connected to the articulated arrangement. A movement of the first pull- and push-rod can lead to the opening or closing of the clamshell gripper. The force transmission unit can, in particular additionally, be configured as a second pull- and push-rod, which is arranged in the shaft and is connected, for example solely, to the clamshell gripper. A movement of the second pull- and push-rod can lead to an angling, in particular in a longitudinal-transverse axial plane, for example the adjustment of any desired angle, in particular between 0° and 90° inclusive, of the clamshell gripper relative to the shaft.

The force transmission unit and the holding unit can be configured as a contiguous shape memory material, in particular a shape memory alloy. The holding unit can be configured as a trap structure, in particular a trap basket. A movement of the force transmission unit can lead to the trap structure, in particular the trap basket, moving out of the shaft and unfolding, or to the trap structure, in particular the trap basket, moving back into the shaft and folding.

The trap structure, in particular the trap basket, can have two individual and three contiguous splines. The at least two, in particular ablation, electrodes and/or (in addition to the at least two electrodes) at least three, in particular measuring, electrodes can be integrated in the splines, in particular in the contiguous splines. The three contiguous splines can have approximately the shape of two stadium-shaped splines joined together at their longitudinal sides.

According to a second aspect, a system for laparoscopic ablation, in particular perivascular neuromodulation, is proposed. The system has a device according to the first aspect. The system has a signal generator arrangement which is connected or can be connected to the device. The system has a control and evaluation unit which is connected or can be connected to the device and/or to the signal generator arrangement.

The signal generator arrangement can have a first signal generator for generating a first signal, in particular a radiofrequency (RF) signal, and a second signal generator for generating a second signal, in particular a signal for pulsed field ablation.

This has the advantage that a combination of RF signals and pulsed field ablation is made possible in one system, and the overall procedure of laparoscopic ablation is shortened. Conventionally, two separate systems, which use either RF signals or pulsed field ablation and must alternately be introduced into a patient's thorax, are used for this purpose.

The control and evaluation unit can be adapted to activate the first signal generator and/or the second signal generator to deliver a signal.

The control and evaluation unit can be configured to activate the first signal generator and/or the second signal generator to deliver a signal in dependence on at least one electrode temperature. The control and evaluation unit can be configured to activate the first signal generator to deliver a signal if the electrode temperature falls below a temperature limit value. The control and evaluation unit can be configured to activate the second signal generator to deliver a signal if the electrode temperature assumes or exceeds the temperature limit value.

The control and evaluation unit can be adapted to apply an electric current to the at least two electrodes and/or to measure a voltage via the at least three electrodes, wherein a nerve activity of the tissue and/or the local tissue impedance can be determined from the electric current and the electric voltage.

According to a third aspect, a method for extravascular, perivascular and/or perineural, neuromodulation is proposed. The method comprises inserting an applicator into a patient. The method comprises positioning a distal end of the applicator at one or more (e. g. two or more) vessels and/or tissues and/or nerves of the patient. The method comprises positioning the distal end of the applicator at the one or more vessels and/or tissues and/or nerves of the patient such that one or more, e. g. at least two, electrodes of the applicator are in contact, e. g. extravascularly, perivascularly and/or perineurally, with a perimeter of the one or more vessels and/or tissues and/or nerves. The method comprises carrying out a denervation by delivering energy via the one or more, e. g. at least two, electrodes. The delivery of energy is effected on the basis of a protocol which has at least one pulse of a pulsed field ablation (PFA). The method comprises withdrawing the applicator.

The method may comprise re-inserting an/the applicator to adjust to anatomical variance.

At least one of the one or more electrodes may be arranged on the applicator. According to a first example, at least two electrodes may be arranged at the applicator and may be used for bipolar ablation. According to a second example, at least one electrode may be arranged at the applicator and may be used for monopolar ablation together with a neutral electrode. The neutral electrode may be arranged outside the patient’s body, e. g. on the skin of the body.

In one specific example, there may be two or more vessels and/or tissues and/or nerves.

In the method according to the third aspect an irreversible electroporation may be carried out.

In the method according to the third aspect a reversible electroporation may be carried out.

The distal end of the applicator can alternatively be positioned at one or more tissues, correspondingly to the positioning at one or more vessels and/or tissues or nerves. A generator system can be provided for carrying out the denervation. The generator can be electrically connected to one or more, e. g. at least two, electrodes integrated or provided distally at the applicator / at the distal end of the applicator.

The applicator can be inserted by means of a laparoscopic procedure. In addition or alternatively, the applicator can be withdrawn by means of a laparoscopic procedure.

The laparoscopic applicator can be introduced into a patient's torso by means of a trocar. The applicator can have the trocar. The applicator can further comprise a shaft having a distal end and a proximal end. The distal end can be equipped, for example, with a plurality of electrodes.

The applicator can be inserted by means of an open surgical procedure. In addition or alternatively, the applicator can be withdrawn by means of an open surgical procedure.

After the applicator has been inserted, geometric unfolding of a distal end of the applicator can be initiated. A form of the distal end that conforms to one or more vessels and/or tissues and/or nerves can thus be achieved. Unfolding can be effected by a mechanical, magnetic or material-based controller.

The one or more, e. g. at least two, electrodes of the applicator can be brought into contact with one or more (target) vessels and/or tissues and/or nerves.

The method can further comprise checking a positioning of the applicator by means of a local impedance measurement. For example, positioning of the applicator can be regarded as complete when one or more, e. g. at least two, electrodes are in contact with the vessel or tissue or nerve. The positioning can subsequently be checked.

The protocol can comprise, for example, only PFA pulses.

The protocol can comprise a combination of PFA pulses and radiofrequency (RF) pulses.

The method can further comprise choosing whether the delivery of energy for the denervation is to be carried out on the basis of a protocol by means of PFA pulses or on the basis of a protocol by means of a combination of PFA pulses and RF pulses.

A delivery of energy can be effected once or multiple times at the same or different locations of the one or more vessels and/or tissues and/or nerves. The delivery of energy can be effected by means of PFA pulses or by means of a combination of PFA pulses and RF pulses.

Single or multiple energy delivery may be affected at one or more different locations on two vessels/nerves/tissues and/or on one vessel/nerve/tissue and then, at another location, on at least two vessels/nerves/tissues, or vice versa.

In the method according to the third aspect, neuromodulation occurs by carrying out the denervation. Neuromodulation through a vessel wall is thus not necessary and can be avoided. For example, extravascular, perivascular and/or perineural neuromodulation may occur by carrying out the denervation.

The method can further comprise, before the denervation is carried out, quantifying a conductivity of the one or more vessels and/or tissues and/or nerves by means of at least two stimulation and measuring electrodes. The quantification of the conductivity can be effected via a stimulation and measuring device, which can have stimulation and measuring electrodes arranged distally and proximally to at least one ablation electrode.

The method can further comprise, after the denervation has been carried out, characterising the denervation by means of at least two stimulation and measuring electrodes. The characterisation of the denervation can be effected via a stimulation and measuring device, which can have stimulation and measuring electrodes arranged distally and proximally to at least one ablation electrode.

While the described procedure is being carried out, a temperature at an ablation site chosen for the ablation can be measured. In other words, the method can further comprise measuring the temperature at the chosen ablation site while the procedure is being carried out.

The method may further comprise positioning the distal end of the applicator at the one or more vessels and/or tissues of the patient such that the one or more electrodes of the applicator are in contact, in particular extravascularly, perivascularly and/or perineurally, with a perimeter of the one or more vessels and/or tissues or lie against a perimeter of the one or more vessels and/or tissues. The method may further comprise carrying out a denervation of one or more nerves not being in contact with the one or more electrodes by delivering energy via the one or more electrodes to the one or more nerves not being in contact with the one or more electrodes.

The step of carrying out a denervation of the one or more nerves not being in contact with the one or more electrodes may comprise delivering energy via the one or more electrodes to the one or more nerves not being in contact with the one or more electrodes through the one or more vessels or tissues being in contact with the one or more electrodes. In other words, the method may further comprise carrying out a denervation of the one or more nerves not being in contact with the one or more electrodes by delivering energy via the one or more electrodes to the one or more nerves not being in contact with the one or more electrodes through the one or more vessels and/or tissues being in contact with the one or more electrodes.

The method may further comprise carrying out a denervation of the one or more nerves not being in contact with the one or more electrodes by way of an electrical field. The electrical field may be perpendicular to axes, particularly longitudinal axes, of the one or more nerves. Potentially the electrical field could be angular or even parallel to the nerve axis, as nerve fibers run in different directions around the vessel(s).

The one or more nerves not being in contact with the one or more electrodes may be located between the one or more vessels or may be hidden by other tissue.

The one or more vessels may be or comprise a vein and/or an artery, particularly a renal vein and/or a renal artery. In other words, the one or more vessels may be or comprise only an artery or only a vein or an artery and a vein. For example, the artery can already be separated and the method may therefore already be used without any separation.

Further features, properties, advantages and possible modifications will become clear to a person skilled in the art from the following descriptions, in which reference is made to the accompanying drawings.

### BRIEF DESCRIPTION OF THE DRAWINGS

FIG. 1 is a schematic representation of a biphasic IRE pulse according to a variant of an embodiment.

FIG. 2 is a schematic representation of a pulse protocol with multiple bursts of biphasic pulses according to a variant of an embodiment.

FIG. 3 shows a schematic representation of a procedure protocol, with at least one burst of biphasic IRE pulses combined with at least one RF energy burst 120, according to a variant of an embodiment.

FIG. 4 shows an embodiment of the distal portion of a laparoscopic applicator.

FIG. 5 shows schematic representations for the activation of the above-described electrodes to carry out a neuromodulation procedure.

FIG. 6 shows an embodiment of the distal portion of a laparoscopic applicator.

FIG. 7 shows schematic representations of electrode arrangements in the interior of a suction cup and activation possibilities for carrying out a procedure of neuromodulation.

FIG. 8 shows an embodiment of the distal portion of a laparoscopic applicator.

FIG. 9 shows a further embodiment of the distal portion of a laparoscopic applicator.

FIG. 10 shows a schematic sequence for carrying out laparoscopic neuromodulation using the example of a denervation procedure.

FIG. 11 shows a further embodiment of the distal portion of a laparoscopic applicator.

FIG. 12 shows the further embodiment of the distal portion of FIG. 11 in another view.

FIG. 13 shows a schematic sequence for carrying out a denervation procedure using the embodiment of FIGS. 11 and 12.

### DETAILED DESCRIPTION OF EMBODIMENTS

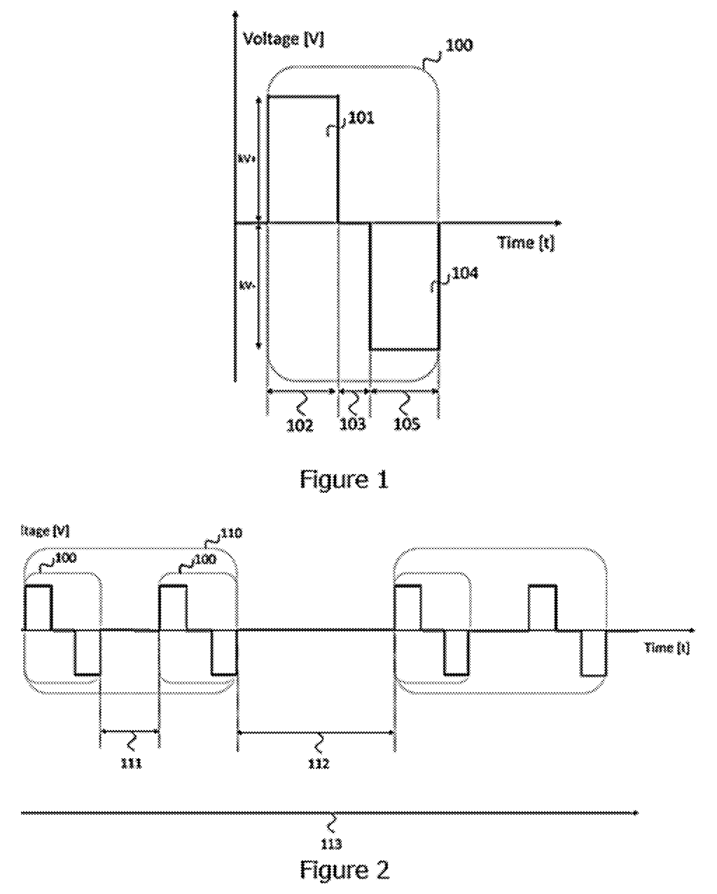

FIG. 1 is a schematic representation of a biphasic IRE pulse (irreversible electroporation) according to an embodiment variant. It shows the voltage V of the biphasic PFA pulse 100 as a function of the time t in an IRE ablation procedure. The present embodiment variant relates to a second signal generator in the form of an IRE generator, which is configured as a voltage source. Consequently, the IRE signals are here described in the form of their voltages. The biphasic IRE pulse 100 comprises a positive pulse 101 and a negative pulse 104, wherein the terms "positive" and "negative" refer to an independently chosen polarity of two electrodes which are activated for the ablation and between which the biphasic pulse is applied. The amplitude of the positive pulse 101 is denoted kV+ and lasts for time 102. Analogously, the amplitude of the negative pulse 104 is denoted kV- and has a duration 105. Between the two pulse phases 101 and 104 there is a delay time 103. Both the two temporal pulse widths 102 and 105 and the amplitudes kV+ and kV- can be configured independently of one another and can therefore vary in an exemplary embodiment of the disclosure.

FIG. 2 is a schematic representation of a pulse protocol with multiple bursts of biphasic pulses according to an embodiment variant. Over the duration of the entire IRE procedure 113, the pulses 100 are delivered in the form of one or more bursts or pulse packets 110. Each burst 110 comprises a defined number N of biphasic pulses 100, wherein the pulses are separated by a time interval 111. There is again a delay time 112 between the delivery of the individual bursts 110.

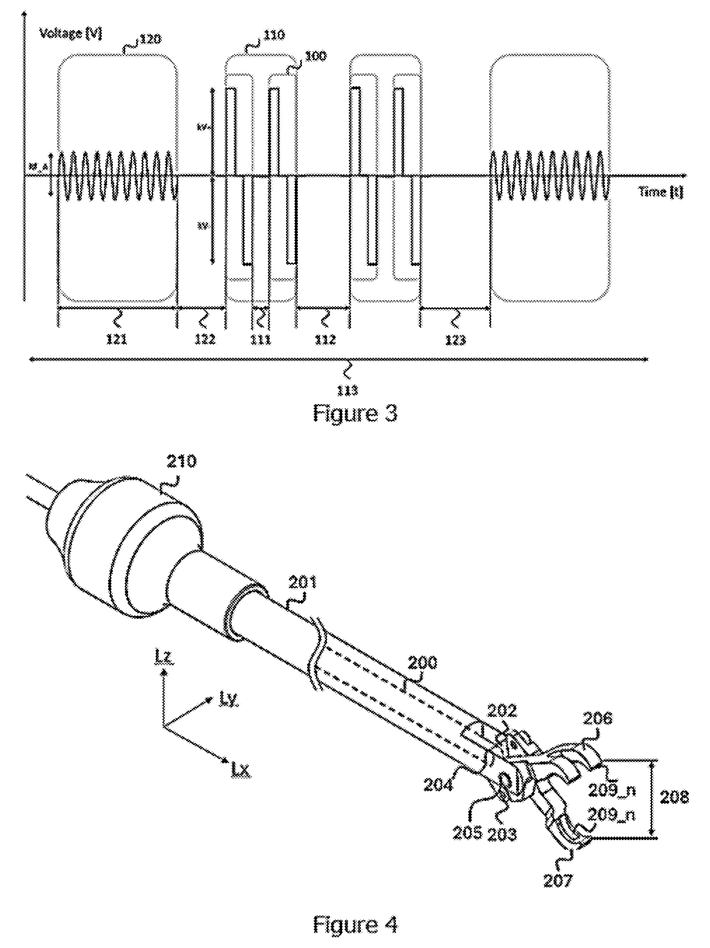

FIG. 3 shows a schematic representation of a procedure protocol with at least one burst of biphasic IRE pulses combined with at least one RF energy burst 120 according to an embodiment variant. Over the duration of the entire combined procedure 113, the RF energy and the IRE pulses are delivered in the form of one or more bursts 120 and 110. Each IRE burst comprises a defined number N of bipolar pulses 100, wherein the pulses are separated by a time interval 111. The RF burst is described by a sinusoidal signal of amplitude RF_A and duration 121. Following an RF burst there is a delay time 122. Both the duration of the RF burst and the subsequent delay time 122 can be adjusted on the basis of the instantaneously measured temperature at the ablation electrodes. There is a delay time 112 between the delivery of the individual IRE bursts 110. There is a delay time 123 between an IRE burst and delivery of a further RF burst.

FIG. 4 shows an embodiment variant of the distal portion of a laparoscopic applicator. The applicator has a shaft 201, which can be inserted through a patient's torso with the aid of a trocar 210. In the interior of the shaft 201 there extends a pull- and pushrod 200, which is movable freely and relative to the shaft along a longitudinal axis Lx. At the distal end of the applicator there is a three-fingered gripper 206, 207 having a two-fingered part 206 and a one-fingered part 207. Each of the three fingers has an inner side, in particular a concave indentation. The three-fingered gripper 206, 207 is adapted to grip a cylindrical tissue structure, the circumference of which is formed about a transverse axis Ly. The cylindrical tissue structure has a longitudinal extent which is oriented parallel to the transverse axis Ly of the laparoscopic applicator. In order to implement the gripping mechanism, the three-fingered gripper 206, 207 is mounted via an articulated arrangement 202, 203, 204, 205 to the outer shaft 201 and also to the inner pull- and pushrod 200 such that a movement of the pull- and pushrod 200 along the longitudinal axis Lx leads to gripping of the three-finger gripper. A gripping distance 208 is thus variably adjustable and accordingly adaptable to different tissue structures. A plurality of electrodes 209_n are integrated in the concave indentations of the three-fingered gripper 206, 207. Each concave indentation has one electrode. Each of the electrodes 209_n is connected via electrical contacts, which extend in the interior of the shaft 201, to an evaluation and control unit (not shown) as well as to the generator system (not shown). These electrodes are each exposed to an external environment, whereas the electrical connections to the control unit and the generator system are insulated with respect to the external environment. In the present embodiment variant, the electrodes are configured as ablation electrodes, whereby laparoscopic ablation is made possible.

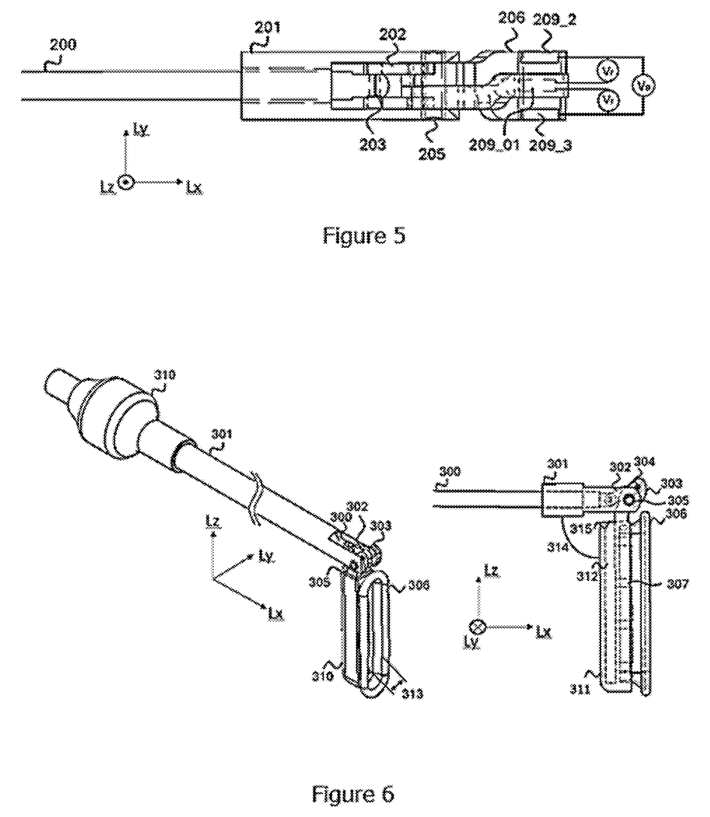

FIG. 5 shows schematic representations for activation of the above-described electrodes 209_n to carry out a neuromodulation procedure. In this embodiment, the electrodes 209_2 and 209_3 are located on the two-fingered part 206 and electrode 209_1 is located on the one-fingered part 207. Activation possibility 1 provides for the generation of an electrical field in the radial direction. To this end, the two voltages Vr are applied between the electrode pair 209_2 and 209_1 as well as between the electrode pair 209_3 and 209_1. When all the electrodes are in contact with a tissue structure, a radially oriented current flow is obtained. In the case of activation possibility 2, a current flow oriented along the transverse axis Ly is induced by application of an electrical field by means of a voltage Va between the electrode pair 209_2 and 209_3. Electrode 209_1 does not have a function here and assumes an electrically floating state.

FIG. 6 shows a further embodiment variant of the distal portion of a laparoscopic applicator. The applicator has an outer shaft 301, which can be inserted through a patient's torso with the aid of a trocar 310. In the interior of the shaft 301 there extends a pull- and pushrod 300, which is movable freely and relative to the shaft 301 along a longitudinal axis Lx. At the distal end of the applicator there is a suction unit 311 having a flexible atraumatic suction cup 306, which is able to establish contact with a cylindrical tissue structure by means of negative pressure. The suction cup 311 is connected via an articulated arrangement 302, 303, 304, 305 to the shaft 301 and to the pull- and pushrod 300 such that an angle 314 between the longitudinal axis Lx of the shaft 301 and the suction unit 311 can freely be adjusted. With the aid of the adjustable angle, both insertion through the trocar 310 can be achieved and optimal contact with the tissue structure can be made possible. In the interior of the suction cup 306 there is a plurality of suction holes 307, which are connected to a suction channel 312. The suction cup has a cup width 313. At the outlet of the suction channel 312 there is a vacuum connection 315, which establishes a connection to an external vacuum pump in order to generate a negative pressure in the interior of the suction cup 306.

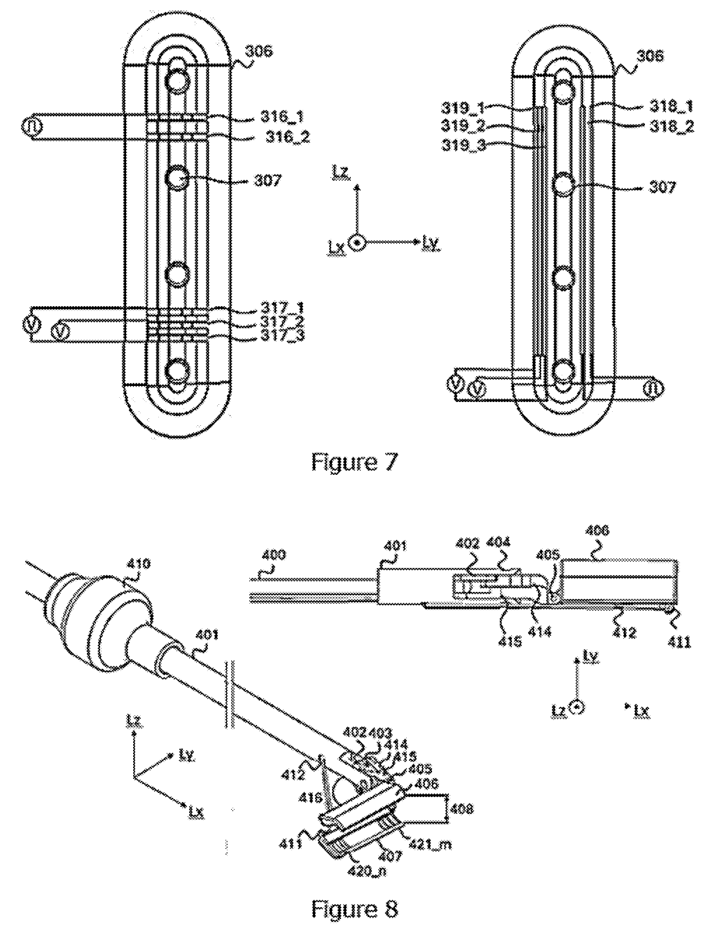

FIG. 7 shows schematic representations of electrode arrangements in the interior of the suction cup 306 and activation possibilities for carrying out a procedure of neuromodulation. In electrode arrangement 1 (on the left) there are two ablation and stimulation electrodes 316_1 and 316_2, as well as three measuring electrodes 371_1, 317_2 and 317_3. All the electrodes are arranged such that they are oriented perpendicular to a longitudinal axis of a suctioned cylindrical tissue structure. Via the two electrodes 316_1 and 316_2, a baseline stimulation of the nerves can be carried out at the start of the procedure by application of a defined electric current to that electrode pair. With the aid of the three measuring electrodes 371_1, 317_2 and 317_3, the stimulus conduction of the nerves is measured while they are simultaneously being stimulated. To that end, two bipolar voltages are detected via the electrode pair 317_1 and 317_2 and via the electrode pair 371_2 and 317_3 and are processed. Following this baseline measurement, bipolar ablation is effected via electrode pair 316_1 and 316_2. In order to characterize the neuromodulation, the nerve conductivity is again measured and analyzed, as described above, following the ablation. The sequence of this procedure is identical in electrode arrangement 2 (on the right). However, it differs in the arrangement of the electrodes within the suction cup 306. In this configuration, all the electrodes are oriented parallel to a longitudinal axis of a suctioned cylindrical tissue structure. The two ablation and stimulation electrodes 318_1 and 318_2 are located on the right of the plurality of suction holes 307, and the measuring electrodes 319_1, 319_2 and 319_3 are located on the left of the plurality of suction holes 307.

FIG. 8 shows an embodiment variant of the distal portion of a laparoscopic applicator. It has an outer shaft 401, which can be inserted through a patient's torso with the aid of a trocar 410. In the interior of the shaft 401 there extends a first pull- and pushrod 400, which is freely movable along a longitudinal axis Lx. At the distal end of the applicator there is a double half-clamshell gripper having a first half-clamshell 406 and a second half-clamshell 407, which each have a concave indentation and are adapted to be able to grip a cylindrical tissue structure. In order to implement the gripping mechanism, the double half-clamshell gripper is mounted via an articulated arrangement 402, 403, 404, 405, 414, 415 to the outer shaft 401 as well as to the inner pull- and push-rod 400, such that a movement of the pull- and push-rod 400 along the longitudinal axis Lx leads to clamping of the first half-clamshell 406 and of the second half-clamshell 407. A clamping distance 408 that is achieved is thus variably adjustable and adaptable to different tissue structures. In the interior of the shaft 401 there extends a second pull- and pushrod 412, which is connected to the first half-clamshell 406 and to the second half-clamshell 407 such that an angle 416 between the longitudinal axis Lx of the shaft 401 and the double half-clamshell gripper can be adjusted. With the aid of the adjustable angle 416, both insertion through the trocar 410 can be achieved and optimal contact with the tissue structure can be made possible. On the concave indentations of the first half-clamshell 406 and of the second half-clamshell 407 there are a plurality of ablation and stimulation electrodes 420_n as well as measuring electrodes 421_m, which are individually connected via electrical contacts, which extend in the interior of the shaft 401, to the evaluation and control unit as well as to the generator system (both not shown). These electrodes are each exposed to the external environment, while the electrical connections to the control unit and to the generator system are insulated from the external environment. Using the example of two ablation and stimulation electrodes and three measuring electrodes, activation is effected analogously to the description in FIG. 7.

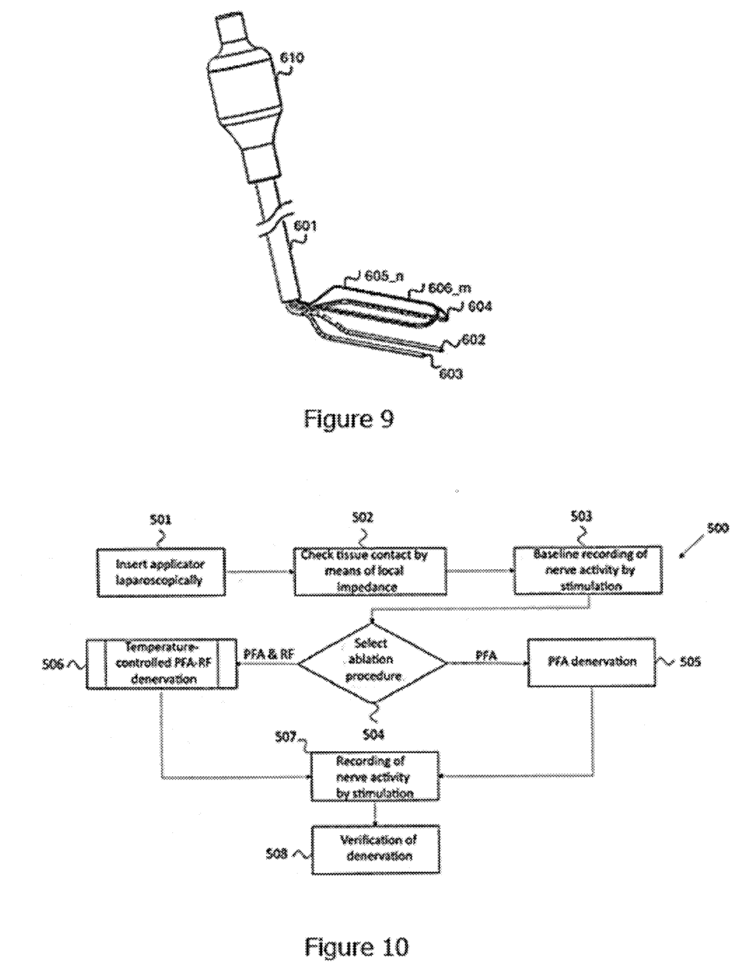

FIG. 9 shows a further embodiment variant of the distal portion of a laparoscopic applicator. The applicator has an outer shaft 601, which can be inserted through a patient's torso with the aid of a trocar 610. In the interior of the shaft 601 there extends a trap structure, for example a trap basket, for example made of a shape memory material, which is composed of three contiguous splines 604 and two individual splines 602 and 603. The entire trap structure is resilient, such that it can be pulled completely into the interior of the shaft 601. It is adapted to be able to grip and clasp, in the state shown, a cylindrical tissue structure. On the three contiguous splines 604 there are in each case a plurality of stimulation and ablation electrodes 605_n as well as a plurality of measuring electrodes 606_m. Each of the plurality of electrodes 605_n, 606_m is individually connected via electrical contacts, which extend in the interior of the shaft 601, to the evaluation and control unit as well as to the generator system (both not shown). The electrodes are each exposed to the external environment, while the electrical connections to the evaluation and control unit and to the generator system are insulated from the external environment. Using the example of two ablation and stimulation electrodes and three measuring electrodes, activation is effected analogously to the description in FIG. 7.

FIG. 10 shows a schematic sequence 500 for carrying out a laparoscopic neuromodulation using the example of a denervation procedure. One or more of the steps described in relation to FIG. 10 can be carried out using a laparoscopic applicator as has been described by way of example in relation to FIGS. 4, 6, 8 and 9. In the first step 501, an applicator system, in particular a laparoscopic applicator, is inserted laparoscopically into the patient. As described by way of example in relation to FIGS. 4, 6, 8 and 10, the laparoscopic applicator can have a trocar by means of which a shaft of the laparoscopic applicator can be inserted into a patient's torso. After the applicator has been positioned, it is checked, by measurement of the local impedance at the ablation electrodes, whether the electrodes have sufficiently good contact with the tissue (step 502). In the next step 503, the starting value of the nerve conductivity is recorded, for example by the mechanisms described above in relation to FIG. 7. For example, by applying a defined electric current to an electrode that is distal or proximal to an ablation electrode, a stimulation pulse can be delivered to one or more nerves. A resulting bipolar voltage is measured at measuring electrode pairs while the nerve is simultaneously being stimulated and is used to characterize the nerve conductivity. Then, in step 504, it is possible to choose between two types of procedure or input options: denervation by means of pulsed field ablation (PFA) or a combination procedure of PFA and RF energy. In the case of PFA denervation (step 505), the procedure, in particular a delivery of energy, is effected on the basis of an exemplary protocol as described in FIG. 2 by means of biphasic IRE pulses. If a PFA-RF combination procedure 506 is chosen, the procedure, in particular a delivery of energy, is effected on the basis of a combination protocol as described by way of example in FIG. 3. Independently of the choice of the denervation procedure, the nerve activity is then recorded again 507 in order to characterise the denervation 508 as described in FIG. 7, FIG. 8 and FIG. 9.

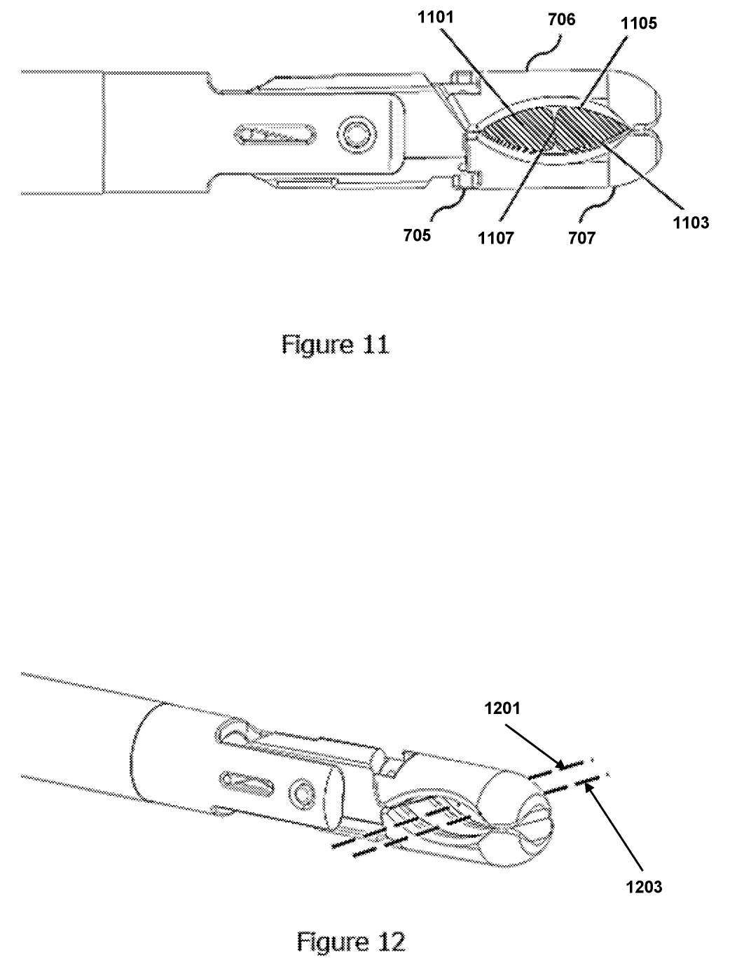

FIG. 11 shows an embodiment variant of the distal portion of a laparoscopic applicator. The applicator has a shaft 701, which can be inserted through a patient's torso e. g. with the aid of a trocar. The applicator has a holding unit 705, which is arranged at the distal end of the shaft. The holding unit 705 is adapted to hold and release one or more vessels and/or tissues and/or nerves. In this particular example, the holding unit 705 is configured as a gripping unit which is adapted to grip and release one or more vessels and/or tissues and/or nerves. For holding, e. g. gripping, the one or more vessels and/or tissues and/or nerves, the applicator has two holding elements or sections 706, 707, e. g. gripping elements or sections. The two holding elements or sections can be opened and or closed as described, for example previously with respect to the three-finger gripper of FIG. 4.

Each of the two holding elements 706, 707 has an inner side, in particular a concave indentation, to hold, e. g. grip, the one or more vessels and/or tissues and/or nerves. As shown in FIG. 11 by way of example, the two holding elements 706, 707 are configured to hold two vessels. The two vessels in FIG. 11 are, by way of example, a vein 1101 and an artery 1103, e. g. the renal vein and the renal artery. In FIG. 11, the nerve fibers running along the artery are shown at 1105 and the nerve fibers located between the artery and the vein and not in direct contact with electrodes are shown at 1107.

In the example shown in FIG. 12, the axis 1201 of the vein and the axis 1203 of the artery are at least substantially parallel to each other. Further, both vessels, particularly both axes 1201, 1203 of the vessels (vein and artery, respectively), are at least substantially parallel to the holding, e. g. gripping, axis of the applicator.

Each of the two holding elements 706, 707 may include one or more electrodes. For example, each of the concave indentations may have one or more electrodes. In this way, a plurality of electrodes, such as electrodes 209_n as described with respect to FIG. 4, may be integrated in/into the two holding elements 706, 707, e. g. the concave indentations of the two holding elements 706, 707. Each of the electrodes 209_n may be connected via electrical contacts, which extend in the interior of the shaft 201, to an evaluation and control unit (not shown) as well as to the generator system (not shown). These electrodes are each exposed to an external environment, whereas the electrical connections to the control unit and the generator system are insulated with respect to the external environment. In the present embodiment variant, the electrodes are configured as ablation electrodes, whereby laparoscopic ablation is made possible.

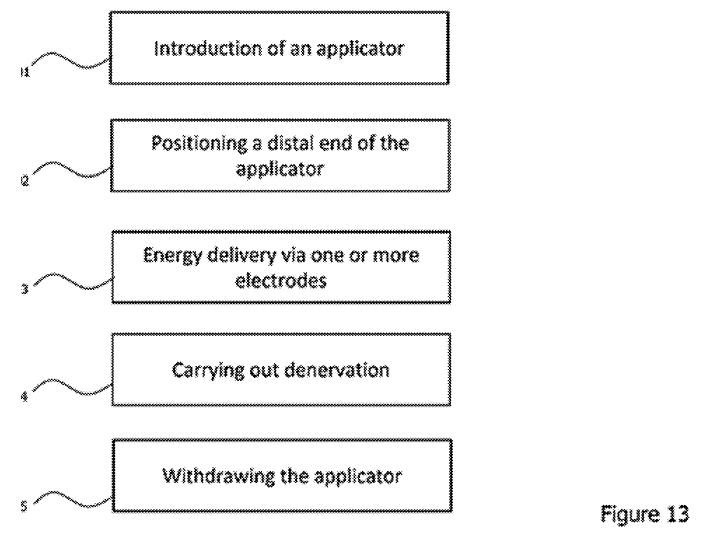

An example of such a laparoscopic ablation/procedure is now described with respect to FIG. 13.

In step S1301, an applicator, such as the applicator shown in FIGS. 11 and 12, is introduced or inserted into a patient. In case of a laparoscopic approach, as described with respect to FIG. 13 by way of example, the applicator is a laparoscopic applicator. In the case of an open surgical approach the applicator may alternatively be another applicator with a grasping distal end.

In step S1302, the distal end of the applicator is positioned (in the patient). More particularly, the distal end is positioned such that it comes or is in contact with one or more, e. g. multiple, tissues and/or vessels and/or nerves.

In step S1303, energy delivery (via a PFA pulse protocol, a RF pulse protocol or a combination thereof) is performed via one or more electrodes, e.g., via one electrode or at least two electrodes, positioned in or at the distal end. The one or more electrodes may be in, e. g. direct, contact with one or more, i. e. multiple, tissues and/or vessels and/or nerves to be ablated. Energy can be delivered at multiple positions along the encompassed vessels. For this purpose, the distal end may be moved and/or electrodes arranged at different positions may be used. Depending on the location and position of the vessels and/or tissues and/or nerves, the energy delivery and/or the direction of the electric field can be controlled via the activation of multiple electrodes. This means that nerves located between vessels, particularly between the artery and vein as shown in FIGS. 11 and 12, or obscured by other tissue and not in direct contact with the ablation electrodes, can be ablated by an electric field oriented, at least in sections, perpendicular or in parallel to their respective axis.

The energy delivery may result in irreversible or reversible denervation in step S1304. The denervation is based on a sequence of PFA pulses, a sequence of RF pulses, or a sequence combining both PFA and RF pulses.

In step S1305, the applicator is withdrawn out of or from the patient.

One advantage of the method described above is that only a single applicator is required for different anatomical variations — regardless of whether vessel separation is necessary. Essentially, there are two scenarios:
1. When inserting the applicator into the patient, it may become apparent that no surgical separation of the vessels is required because the vessels are already anatomically separated. In this case, the applicator encloses only a single vessel, e.g., an artery.
2. When inserting the applicator into the patient, it may become apparent that the vessels are connected. In this case, the same applicator can be used to grasp both vessels simultaneously, thereby avoiding the need for surgical separation.

Overall, the concept is to ensure that the surgeon is prepared for any anatomical variation. In the second scenario, additional advantages include time savings, no need for additional physician training, and reduced trauma to the vein.

In the following, some specifics are described which can be implanted in combination with all methods, method details and devices explained above. More particularly, these specifics are at least partially explained with respect to the two vessels held by the holding unit 705 in FIG. 11, which might be or include the renal vein and renal artery. In case the two vessels are or include the renal vein and the renal artery, the renal artery and the renal vein are encompassed together using the laparoscopic applicator, without separating the vessels or exposing the renal artery beforehand. This leads to time savings. Further, no specific physician training is required. Still, risk is minimized. Further, one advantage resides in using non-thermal energy with a new applicator design that is applicable independent from anatomical variation (no extra step for surgical separation needed, vein is not at risk of damage because of non-thermal energy...).

During the encompassing or encircling process, due to the specific shape of the distal end of the holding unit, e. g. the grasper, vessels — particularly the renal artery and renal vein — are positioned at least substantially parallel next to each other, resulting in the largest possible surface area for ablation. Energy delivery occurs while multiple vessels, e. g. the renal vein and renal artery, are encompassed. In addition, either at least PFA energy or a combination of PFA and RF energy is delivered in order to reach tissue regions (between the two vessels) without causing damage to the vessels through excessive energy delivery.

While some nerve fibers run along the renal artery, other nerve fibers are located outside the renal arterial wall, mainly in the adventitia of the renal artery and in its perivascular compartment. Few or no nerve fibers run around the renal vein. During the delivery of PFA pulses (to the artery and/or vein), the electric field can also reach nerves located at the interface between artery and vein, e. g. nerve fibers located between the artery and the vein. Thus, nerve tissue can be reached and denervated via the electric field even without direct exposure of the renal artery, i.e., without direct contact of the nerve tissue (outside the renal arterial wall) with/to the ablation electrodes. Alternatively, anatomically variable side branches and/or a number of interconnected vessels can also be encompassed using the same applicator.

With respect to renal vessels, although there is an anatomic variability on how the arteries and veins are going into the kidney, both can be treated with the proposed concept. It may be possible to treat only the artery, if they are already separated, or treat both if the bundle is attached.

In addition to an advantageous applicator, a laparoscopy-based neuromodulation of nerves by a combined procedure consisting of successive IRE and radiofrequency (RF) sequences is described herein. In this manner, the tissue conductivity can be increased in a targeted manner while the temperature is monitored continuously. The success of the irreversible electroporation can thus be maximised and at the same time the occurrence of potentially damaging high current densities can be reduced. By using the applicator, ablation does not have to be carried out through the vessel wall.

What is claimed is:

1. Method for extravascular, perivascular and/or perineural neuromodulation,
comprising:
inserting an applicator into a patient;
positioning a distal end of the applicator at one or more vessels and/or tissues and/or nerves of the patient such that one or more electrodes of the applicator are in contact with a perimeter of the one or more vessels and/or tissues and/or nerves or lie against a perimeter of the one or more vessels and/or tissues and/or nerves;
carrying out a denervation by delivering energy via the one or more electrodes, wherein the delivery of energy is affected on the basis of a protocol which has at least one pulse of a pulsed field ablation (PFA); and
withdrawing the applicator.

2. Method according to claim 1, wherein an irreversible electroporation is carried out.

3. Method according to claim 1, wherein a reversible electroporation is carried out.

4. Method according to claim 1, wherein the applicator is inserted and/or withdrawn by a laparoscopic procedure.

5. Method according to claim 1, wherein the laparoscopic applicator is inserted into a patient's torso by means of a trocar.

6. Method according to claim 1, wherein the applicator is inserted and/or withdrawn by means of an open surgical procedure.

7. Method according to claim 1, wherein, after the applicator has been inserted, geometric unfolding of a distal end of the applicator is initiated and a form of the distal end that conforms to one or more vessels or tissues or nerves is thus achieved.

8. Method according to claim 1, wherein the one or more electrodes of the applicator are brought into contact with one or more target vessels and/or target tissues and/or target nerves.

9. Method according to claim 1, further comprising:
checking a positioning of the applicator by means of a local impedance measurement.

10. Method according to claim 1, wherein the protocol comprises only PFA pulses.

11. Method according to claim 1, wherein the protocol comprises a combination of PFA pulses and radiofrequency (RF) pulses.

12. Method according to claim 1, further comprising:
choosing whether the delivery of energy for the denervation is to be carried out on the basis of a protocol by means of PFA pulses or on the basis of a protocol by means of a combination of PFA pulses and RF pulses.

13. Method according to claim 1, wherein a delivery of energy is affected once or multiple times at the same or different locations of the one or more vessels and/or tissues and/or nerves.

14. Method according to claim 1, wherein l neuromodulation occurs by carrying out the denervation.

15. Method according to claim 1, further comprising:
before the denervation is carried out, quantifying a conductivity of the one or more vessels and/or tissues and/or nerves by means of at least two stimulation and measuring electrodes.

16. Method according to claim 1, further comprising:
after the denervation has been carried out, characterising the denervation by means of at least two stimulation and measuring electrodes.

17. Method according to claim 1, further comprising:
measuring a temperature at a chosen ablation site.

18. Method according to claim 1, wherein the method comprises:
positioning the distal end of the applicator at one or more vessels and/or tissues of the patient such that the one or more electrodes of the applicator are in contact with a perimeter of the one or more vessels and/or tissues or lie against a perimeter of the one or more vessels and/or tissues; and
carrying out a denervation of one or more nerves not being in contact with the one or more electrodes by delivering energy via the one or more electrodes to the one or more nerves not being in contact with the one or more electrodes.

19. Method according to claim 18, wherein the step of carrying out a denervation of the one or more nerves not being in contact with the one or more electrodes comprises delivering energy via the one or more electrodes to the one or more nerves not being in contact with the one or more electrodes through the one or more vessels or tissues being in contact with the one or more electrodes.

20. Method according to claim 19, wherein the method comprises carrying out a denervation of the one or more nerves not being in contact with the one or more electrodes by way of an electrical field which is, at least in sections, perpendicular or parallel to axes of the one or more nerves.

21. Method according to claim 18, wherein the one or more nerves not being in contact with the one or more electrodes are located between the one or more vessels or are hidden by other tissue.

22. Method according to claim 21, wherein the one or more vessels are a vein and an artery.
